(19) United States
(12) Patent Application Publication
(10) Pub. No.: US 2006/0020224 A1
(43) Pub. Date: Jan. 26, 2006

(54) Intracranial pressure monitoring system

(76) Inventor: Mark A. Geiger, Ventura, CA (US)

Correspondence Address: SHUMAKER & SIEFFERT, P. A., 8425 SEASONS PARKWAY, SUITE 105, ST. PAUL, MN, 55125, US

(21) Appl. No.: 10/976,164

(22) Filed: Oct. 28, 2004

Related U.S. Application Data
(60) Provisional application No. 60/589,347, filed on Jul. 20, 2004.

(51) Int. Cl. A61B 5/00

(52) U.S. Cl. 600/561; 128/903

(57) ABSTRACT

The disclosure is directed to a system and method for monitoring ICP within a patient on a continuous or periodic basis over an extended period of time. In some situations, a care-giver may want to record ICP measurements over a longer period of time to obtain trend data. A system for monitoring ICP includes a shroud-like, inductive power transmitted element designed to surround at least a substantial portion of a patient's head and power an implanted ICP monitor. The shroud-like element may be a table-mounted device that arcs over the width of the table or bed, providing room for the patient's head.

This application claims the benefit of U.S. provisional application No. 60/589,347, filed Jul. 20, 2004, the entire content of which is incorporated herein by reference.

### FIELD OF THE INVENTION

The invention relates to medical devices and, more particularly, devices for draining cerebral spinal fluid.

### BACKGROUND

Hydrocephalus is an excess accumulation of cerebrospinal fluid (CSF) in the ventricles of the brain. This fluid, which protects, nourishes and cleanses the brain and spinal cord, is manufactured daily in the ventricles. Buildup of CSF occurs when the fluid cannot flow freely throughout the ventricles and the central nervous system due to various forms of blockage. Except in very rare cases, hydrocephalus is a life-long condition that can only be controlled, not cured, through medical intervention. There are a number of accepted treatments available for hydrocephalus, most of which involve the surgical implantation of a shunt. The shunt diverts CSF from the brain ventricles to another part of the patient's body.

Elevated intracranial pressure (ICP) can be a problem for patients suffering from chronic hydrocephalus, as well as patients with brain injuries or other diseases that cause an acute accumulation of CSF. An ICP monitor provides an indication of ICP so that a care-giver can intervene in the event ICP becomes too high. For example, a care-giver may adjust a valve associated with a shunt, administer medication or take other action to relieve elevated ICP levels. An external ICP monitor may be coupled to a catheter that extends into the cranium. Alternatively, the ICP monitor may form part of an implanted ventricular shunt catheter, or be implanted independently of the ventricular shunt catheter.

Implantable telemetric ICP monitors are equipped to sense ICP and transmit wireless signals representing the sensed ICP level. Typically, an implantable ICP monitor does not include a battery or data storage. Instead, the ICP monitoring device is ordinarily powered inductively by an external device, and provides an instantaneous “snap-shot” of ICP at a particular point in time. In this case, the ICP includes a pressure sensor, monitoring circuitry, a wireless transmitter, and an inductive power interface. The inductive power interface receives inductively coupled energy and generates power for the sensor and transmitter.

Table 1 below lists documents that disclose implantable telemetric ICP monitors. U.S. Pat. No. 4,519,401 to Ko et al. describes a battery-powered implantable ICP monitor with low power pressure sensing circuitry and wireless telemetry. U.S. Pat. No. 6,113,553 to Chubbuck describes an implantable, inductively powered ICP monitor providing wireless telemetry. U.S. Pat. No. 6,533,733 to Ericson et al. describes an implantable ICP monitor that can be powered by an internal power source or an inductively coupled, external power source. U.S. Pat. No. 6,248,080 to Miesel et al. describes an implantable, battery powered ICP monitor with wireless telemetry.

TABLE 1

| Patent | | |
|---|---|---|
| Number | Inventors | Title |
| 4,519,401 | Ko et al. | Pressure telemetry implant |
| 6,113,553 | Chubbuck | Telemetric intracranial pressure monitoring |
| | | system |
| 6,533,733 | Ericson | Implantable device for in-vivo intracranial and |
| | et al. | cerebrospinal fluid pressure monitoring |
| 6,248,080 | Miesel | Intracranial monitoring and therapy delivery |
| | et al. | control device, system and method |

All documents listed in Table 1 above are hereby incorporated by reference herein in their respective entireties. As those of ordinary skill in the art will appreciate readily upon reading the Summary, Detailed Description and claims set forth below, many of the devices and methods disclosed in the patents of Table 1 may be modified advantageously by using the techniques of the present invention.

### SUMMARY OF THE INVENTION

In general, the invention is directed to a system and method for monitoring ICP within a patient on a continuous or periodic basis over an extended period of time using an inductive power element that extends over a substantial portion of a patient's head to inductively power an implanted ICP monitor. The inductive power element also may serve as a telemetry antenna to receive wireless telemetry signals transmitted by the ICP monitor. The inductive power element may be shroud-like, and define an opening to receive at least a portion of the patient's head.

Various embodiments of the present invention provide solutions to one or more problems existing in the prior art with respect to prior art systems for ICP monitoring. These problems include the inconvenience and discomfort associated with external, catheter-based ICP monitors, and the intermittent nature of measurements obtained by conventional implanted telemetric ICP monitors. Typically, an implantable, telemetric ICP monitor does not include a battery or data storage, and instead must be powered inductively by an external device. Hence, an ICP monitor may provide only an instantaneous “snap-shot” of ICP at a particular point in time at which the IPC monitor is powered. Consequently, it is difficult for a care-giver to obtain continuous ICP measurements over an extended period of time using an implanted, telemetric ICP monitor. As a further problem, a care-giver is unable to detect significantly elevated ICP levels that may occur between intermittent measurements. The inability to detect elevated ICP levels between measurements can expose the patient to health risks.

Various embodiments of the present invention are capable of solving at least one of the foregoing problems. When embodied in a system or method for monitoring vital signs, the invention includes features that facilitate the continuous or periodic measurement of ICP over an extended period of time without the need for a persistent, catheter-based ICP monitor. In this manner, the invention enables a care-giver to obtain measurements from an implanted ICP monitor on a more continuous basis. The ability to obtain measurements on a more continuous basis permits generation and analysis of a larger body of data that may be useful in diagnosis and care decisions. For example, in some situations, a care-giver may want to record ICP measurements over a longer period of time to obtain trend data. In addition, continuous or periodic measurements permit the detection of elevated levels of ICP, and the delivery of therapy to relieve or otherwise reduce health risks posed by such levels.

In accordance with the invention, a system for monitoring ICP includes an element designed to extend over at least a substantial portion of a patient's head. In some embodiments, the element may be a table- or bed-mounted device that arcs over the width of the table or bed, providing room for the patient's head. In some cases, the patient may sleep with his head within an opening defined by the element.

The element is electrically conductive and transmits inductive energy to power an ICP monitor implanted in the patient's head. In addition, the element may serve as an antenna to telemetrically receive information transmitted by the power ICP monitoring device. In this manner, the system can both power the ICP monitor and receive ICP information on a substantially continuous or periodic basis. The element may define an opening sufficiently small to permit reliable inductive power transfer and telemetry with the implantable ICP monitor, yet large enough to comfortably accommodate the patient's head.

An external monitor may be provided, e.g., at the patient's bedside, to receive and process the measurement information received by the element from the implanted ICP monitoring device. The external monitor may be capable of storing received information, and may include ports for download, display or other output of the information. In some embodiments, the external monitor may be a vital signs monitor that accepts inputs from a variety of different vital signs sensors.

In one embodiment, the invention provides a system for monitoring intracranial pressure (ICP), the system comprising an implantable ICP monitor for implantation in a head of a patient, an inductive power transmitting element sized to extend over at least a substantial portion of the head of a patient and inductively power the ICP monitor, and an external monitor to receive the transmitted ICP signal from the ICP monitor.

In comparison to known techniques for monitoring ICP, various embodiments of the invention may provide one or more advantages. For example, the invention enables a care-giver to obtain ICP measurement information over an extended period of time, and even when the patient is sleeping. The ability to obtain ICP measurements on a continuous or periodic basis allows a care-giver to obtain a valuable body of information, and permits the care-giver to detect potentially dangerous ICP levels. Consequently, the invention may contribute to improved patient care. At the same time, an element as described herein can be constructed in a manner that provides the patient with comfort and convenience, relative to catheter-based ICP monitors.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is a side view illustrating an ICP monitoring system having a shroud-like inductive power and telemetry element in conjunction with a patient, in accordance with an embodiment of the invention.

FIG. 2 is a rear view illustrating the ICP monitoring system of FIG. 1.

FIG. 3 is a perspective diagram of an ICP monitoring system as shown in FIG. 1 in conjunction with a patient bed.

FIG. 4 is an enlarged view of a portion of the shroud-like element of FIGS. 1-3, in accordance with an embodiment of the invention.

FIGS. 5 and 6 are rear views of the ICP monitoring system of FIG. 1 illustrating alternative shroud designs.

FIG. 7 is schematic diagram of an ICP monitor implanted in the cranium of a patient.

FIG. 8 is a cross-sectional side view of the implantable ICP monitor of FIG. 7.

FIG. 9 is a block diagram illustrating an ICP monitoring system in accordance with an embodiment of the invention.

FIG. 10 is a block diagram illustrating an implantable ICP monitor.

FIG. 11 is a block diagram illustrating an external power/telemetry unit to power and communicate with an implanted ICP monitor.

FIG. 12 is a flow diagram illustrating an ICP monitoring method in accordance with an embodiment of the invention.

### DESCRIPTION OF THE PREFERRED EMBODIMENTS

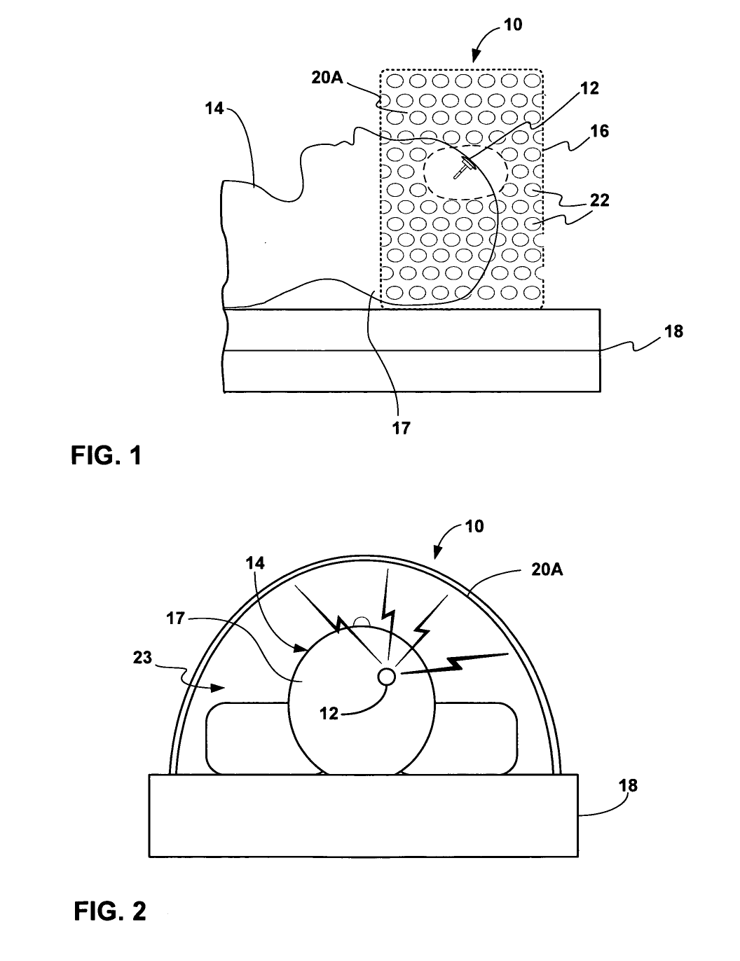

FIG. 1 is a side view illustrating an ICP monitoring system 10 in conjunction with a patient 14, in accordance with an embodiment of the invention. As shown in FIG. 1, system 10 includes an implantable ICP monitor 12, which has been implanted in the cranium of patient 14 to obtain ICP measurements. In addition, system 10 includes a shroud-like element 16 that extends over a substantial portion of the patient's head, including the portion in which ICP monitor 12 is implanted. As will be described, shroud-like element 16 may serve as both an inductive power transmitting element and a telemetry receiver antenna.

In some embodiments, shroud-like element 16 defines a tube- or arc-like opening to receive the patient's head 17. Shroud-like element 16 may be mounted to a bed 18, and is constructed, at least partially, from an electrically conductive frame 20A. In other embodiments, shroud-like element 16 may be mounted to a table, chair or other support platform for patient 14.

Electrically conductive frame 20A may include an insulative substrate and an array of wires or conductive traces that form loops of an electromagnetic coil. As an alternative, electrically conductive frame 20A may carry an array of separated electromagnetic coils coupled in common to a source of alternating current. In this case, the individual coils contribute to an overall inductive field. In addition, electrically conductive frame 20A may define an array of apertures 22 such as round or elliptical holes or square or rectangular slots, if desired, to promote ventilation. Apertures 22 also may serve to tune the electromagnetic properties of shroud-like element 16.

Shroud-like element 16 is coupled to an inductive power generator (not shown in FIG. 1) to receive an alternating current (ac) signal, which is generates an electromagnetic signal that is transmitted by the inductive coil or coils of electrically conductive frame 20A of shroud-like device 16 to power implantable ICP monitor 12. Hence, the shroud-like element 16 serves as an inductive power transmitter for the transfer of energy to ICP monitor 12. In addition, electrically conductive frame 20A may serve as an antenna to receive wireless signals transmitted by implantable ICP monitor 12. The wireless signals convey ICP pressure measurements or other operational or status information associated with implantable ICP monitor.

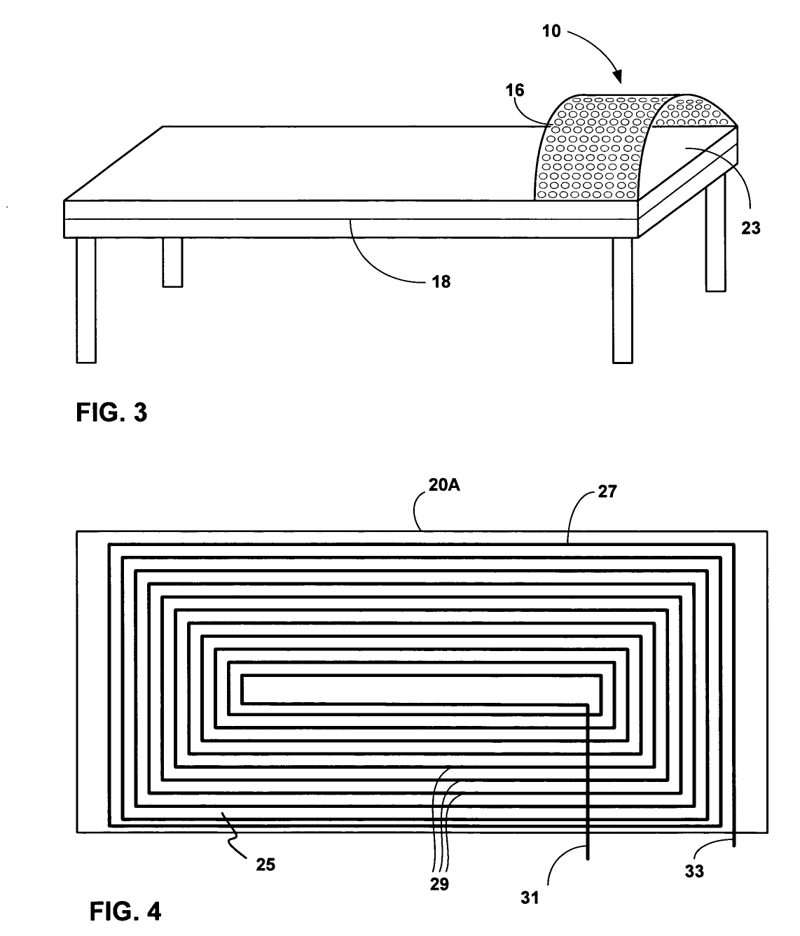

FIG. 2 is a rear view illustrating ICP monitoring system 10 of FIG. 1. FIG. 3 is a perspective diagram of an ICP monitoring system 10 in conjunction with patient bed 18. As shown in FIGS. 2 and 3, electrically conductive frame 20A of shroud-like element 16 may define a tube- or arc-like opening 23 to receive the head 17 of patient 14. Opening 23 of shroud-like device 16 is sized sufficiently small to permit reliable inductive transfer of power from electrically conductive frame 20A to implantable ICP monitor 12, as well as reliable wireless telemetry between the implantable ICP monitor and the electrically conductive frame.

For example, opening 23 may be sized to provide a distance of approximately 1 cm to 10 cm between implantable ICP monitor 12 in head 17 of patient 14 and an interior surface of element 16. In this manner, opening 23 can be sized to balance patient comfort with reliable power transfer and wireless telemetry. In some embodiments, electrically conductive frame 20A may have an adjustable size or height in order to accommodate patients of different sizes.

Electrically conductive frame 20A may be constructed as a continuous sheet of conductive material, or include apertures 22, as described above. As further alternatives, electrically conductive frame 20A may be constructed as a mesh or cage-like assembly, or carry an array of inductive coils. As shown in FIGS. 2 and 3, electrically conductive frame 20A may be substantially hemispherical in shape, but may be subject to other shapes or sizes. A hemispherical, arc-like shape may be advantageous in terms of minimizing the average distance between electrically conductive frame 20A and ICP monitor 12. Also, an arc-like shape may provide more effective coverage when the patient 14 has more than one implanted ICP monitor. However, other shapes for opening 23 may be used, such as rectangular, square, or triangular shapes. As a further alternative, electrically conductive frame 20A may be constructed to extend only partially over the head 17 of patient 14.

In operation, shroud-like element 16 continuously or periodically powers implanted ICP monitor 12, in which case the ICP monitor continuously or periodically transmits signals representative of ICP levels. Shroud-like element 16 receives the signals and couples them to an external monitor (not shown in FIGS. 1-3) for processing, analysis and presentation. The external monitor may provide a continuous or periodic indication of ICP, and may invoke advisory levels at which an ICP measurement may trigger an alarm or other indicator for the attention of a care-giver.

FIG. 4 is an enlarged view of a portion of shroud-like element 16 of FIGS. 1-3, in accordance with an embodiment of the invention. Electrically conductive frame 20A of shroud-like element 16 may be constructed in a variety of ways. In the example of FIG. 4, however, electrically conductive frame 20A is constructed to having an insulative substrate 25 with an inductive coil 27 having a plurality of turns 29 and terminals 31, 33. Turns 29 may be embedded wires or conductive traces, and may be formed from a variety of conductive materials, such as copper, silver or platinum. Insulative substrate 25 may be formed from any of a variety of polymeric, dielectric materials, and may be selected to have desired dielectric properties in some embodiments.

An inductive power generator drives terminals 31, 33 to cause turns 29 of inductive coil 27 to generate electromagnetic energy for transfer to implantable ICP monitor 12. In addition, turns 29 serve to receive telemetry signals from implantable ICP monitor 12. A telemetry circuit is coupled to terminals 31, 33 to process signals received by inductive coil 27 of electrically conductive frame 20A. As an alternative to the single inductive coil 27 of FIG. 4, electrically conductive frame 20A may include an array of individual coils. In either case, shroud-like element 16 emits electromagnetic energy for inductive transfer to implantable ICP monitor 12 and receives telemetry signals from the ICP monitor.

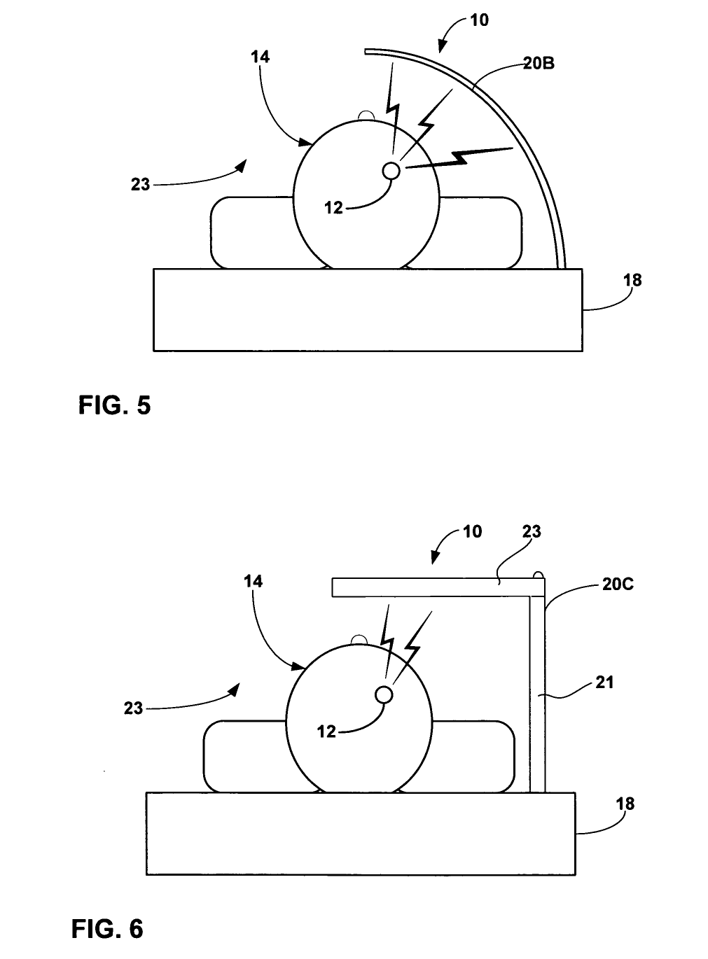

FIGS. 5 and 6 are rear views of the ICP monitoring system of FIG. 1 illustrating alternative designs for shroud-like element 16. As shown in FIG. 5, shroud-like element 16 may have an electrically conductive frame 20B with a quarter-spherical arc that extends only partially over head 17 of patient 14. In the example of FIG. 5, it is desirable to place head 17 of patient 14 in a position at which distance between implanted ICP monitor 12 and electrically conductive frame 20B. As shown in FIG. 6, shroud-like element 16 may have an electrically conductive frame 20C with a right-angled configuration, including a vertical member 21 and a horizontal member 23. Horizontal member 23 extends over head 17 of patient 14. In some embodiments, vertical member 21 may be provided strictly for support of horizontal member 23. In that case, horizontal member 23 serves as an inductive power transmitter and a telemetry antenna.

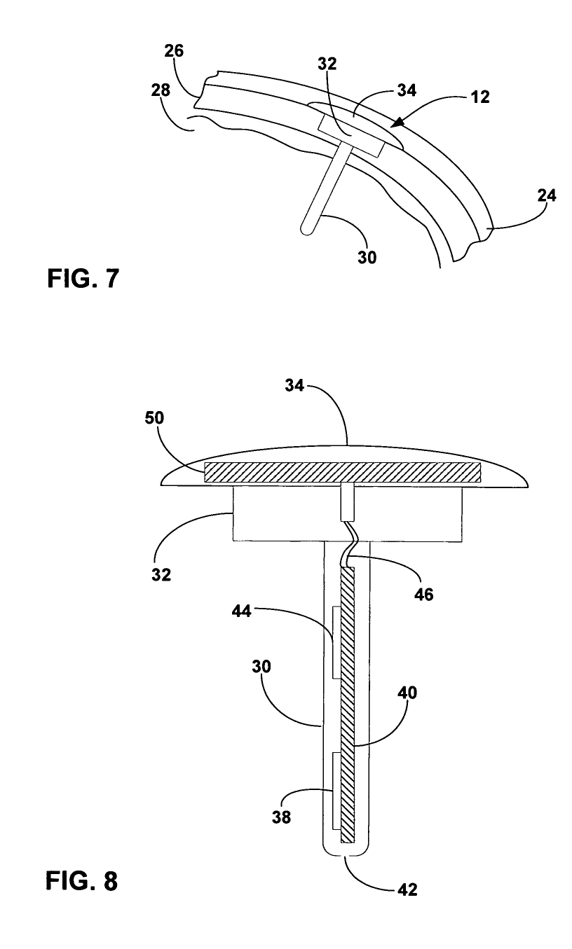

FIG. 7 is schematic diagram of an ICP monitor 12 implanted in the cranium of a patient. ICP monitor 12 may be constructed as a conventional implantable ICP monitor. In some embodiments, ICP monitor 12 may generally conform to a monitor as described in U.S. Pat. No. 6,248,080 to Miesel et al., the entire content of which is incorporated herein by reference. As shown in FIG. 7, ICP monitor 12 is implanted beneath scalp 24, and includes a portion that extends through skull 26 and into brain 28. For example, ICP monitor 12 may include a pressure sensor probe 30, a silicone plug 32 and a cap member 34. Silicone plug 32 fills and substantially seals a burr hole within skull 26. Probe 30 extends inward from silicone plug 32 and penetrates brain 28. Cap 34 rests under scalp 24 and over skull 26.

FIG. 8 is a cross-sectional side view of the implantable ICP monitor 12 of FIG. 7. As shown in FIG. 8, probe 30 may include a pressure sensor 38 carried on a circuit board 40. Pressure sensor 38 may take the form of any of a variety diaphragm sensors, strain gauge sensors, capacitive sensors, piezoelectric sensors, or other sensors used in conventional ICP pressure measurement. Probe 30 may define a hole 42 for fluid communication with the environment with the cranium. Additional circuitry 44 may be provided on circuit board 40 to amplify, filter and process the pressure signal output by pressure sensor 38. In addition, circuitry 44 may be electrically coupled, via conductors 46, to an inductive coil 50 within cap 34. Accordingly, circuitry 44 also may include power generation circuit to convert current induced in coil 50 into operating power, and telemetry circuitry to drive the coil for transmission of signals carrying ICP measurements from pressure sensor 38.

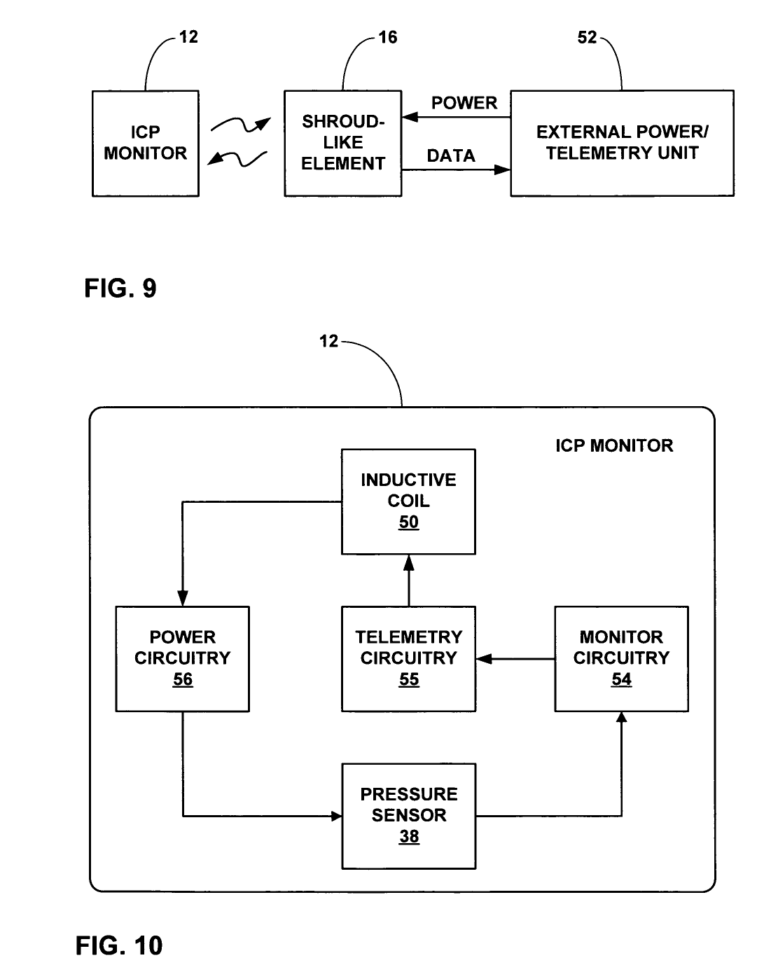

FIG. 9 is a block diagram illustrating an ICP monitoring system 12 in accordance with an embodiment of the invention. As shown in FIG. 9, an external power/telemetry unit 52 generates power to drive shroud-like element 16. Implantable ICP monitor 12 receives power by inductive transfer from shroud-like element 16. In addition, ICP monitor 12 then transmits ICP measurement signals, which are received by shroud-like element 16 as an antenna. External power/telemetry unit 52 receives the ICP measurement signals from shroud-like element 16 for further processing, analysis and presentation to care-givers. In this manner, shroud-like element 16 provides a persistent link between implantable ICP monitor 12 and external power/telemetry unit 52 for continuous or periodic ICP monitoring.

FIG. 10 is a block diagram illustrating an implantable ICP monitor 12. As shown in FIG. 10, implantable ICP monitor 12 includes pressure sensor 38, inductive coil 50, monitor circuitry 54, telemetry circuitry 55 and power conversion circuitry 56. Pressure sensor 38 senses intracranial pressure and generates an ICP measurement signal. The ICP measurement signal may be transmitted substantially in real-time, or buffered within ICP monitor 12 for a period of time. In some embodiments, ICP monitor 12 may support bi-directional communication and may be configured to transmit pressure measurement signals in response to an interrogation request transmitted by external power/telemetry unit 52.

Coil 50 receives electromagnetic energy from shroud-like element 16, which induces current in the coil. Hence, ICP monitor 12 is powered by inductive telemetric transmission of energy. Power conversion circuitry 56 converts current induced in inductive coil 50 into operating power for pressure sensor 38, monitor circuitry 54 and telemetry circuitry 55. For example, power circuit 56 may include an ac/dc conversion circuit, such as a rectifier, that converts the ac current induced in coil 50 into dc operating power. The electromagnetic energy transmitted by shroud-like element 16, and hence the ac current induced in coil 50, may reside within any frequency range suitable for effective inductive transfer of energy, as is known in the art. For example, transmission frequencies of approximately 100 kHz to several MHz may be suitable for inductive telemetric energy transfer, although other frequencies may be used. Wireless signals generated by ICP monitor 12 may reside within the telemetric power frequency range, or any other frequency ranges suitable for reliable communication. In some embodiments, shroud-like element 16 serves as both an integrated power source and signal receiver for ICP monitor 12.

Power conversion circuitry 56 also may include a capacitor or other storage device to store a dc potential as a source of operating power. The capacitor may store energy temporarily to power ICP monitor 12, e.g., only during the time that coil 50 receives energy from shroud-like element 16. Alternatively, a battery may be provide to power ICP monitor 12 over an extended period of time. In some embodiments, power conversion circuitry 56 may generally correspond to similar circuitry described in U.S. Pat. No. 6,731,976 to Penn et al., the entire content of which is incorporated herein by reference.

Monitor circuitry 54 filters, amplifies, and processes the ICP measurement signal, as necessary. Telemetry circuitry 55 then generates telemetry signals for wireless transmission to external power/telemetry unit 52, using inductive coil 50 and shroud-like element 16 as antennas. Hence, inductive coil 50 and shroud-like element 16 serve as inductive transfer elements for purposes of both power transfer and telemetry. Telemetry circuitry 55 includes appropriate amplifier, filtering and modulation circuitry to convert the ICP measurement signal into a telemetry signal.

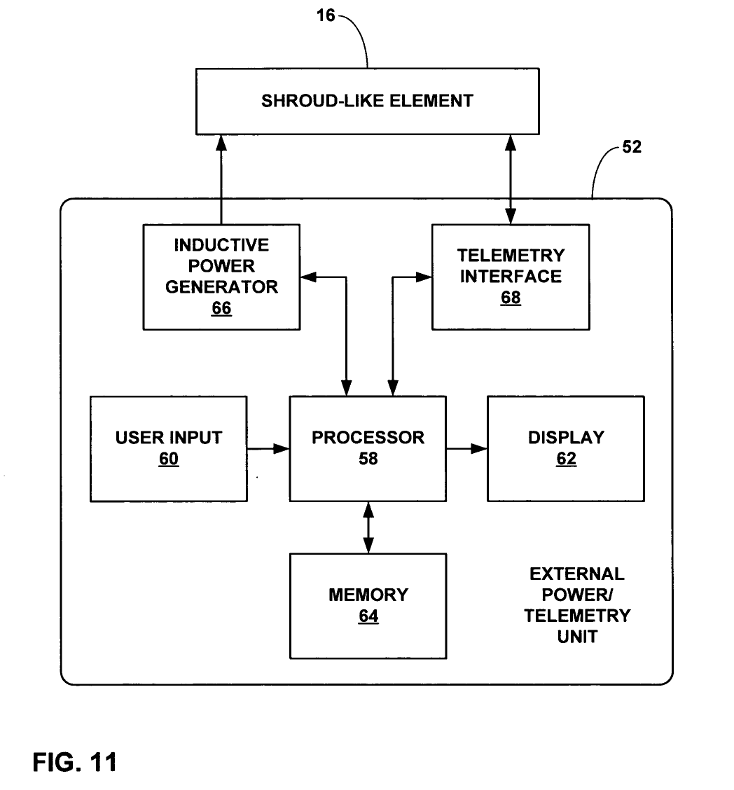

FIG. 11 is a block diagram illustrating an external power/telemetry unit 52 to power and communicate with an implanted ICP monitor 12. As shown in FIG. 11, external power/telemetry unit 52 may include a processor 58, a user input device 60, display 62, memory 64, inductive power generator 66 and telemetry interface 68.

Processor 58 controls the operation of the various components of external power/telemetry unit 52. For example, processor 58 controls inductive power generator 66 and telemetry interface 68, and handles processing and storage of information obtained from implantable ICP monitor 12. Processor 58 may include one or more microprocessors, digital signal processors (DSPs), application-specific integrated circuits (ASICs), field-programmable gate arrays (FPGAs), or other equivalent logic circuitry.

Processor 58 also may accept input from user input device 60, e.g., to select different formats, or time or amplitude scales, for presentation of ICP information on display 62. Display 62 may include any of a variety of different displays, such as a liquid crystal display (LCD), plasma display, or cathode ray tube (CRT) display. In addition, processor 58 may archive ICP information within memory 64 for retrieval or transmission to other devices, such as remote monitors distributed within a network.

Memory 64 may include any magnetic, electronic, or optical media, such as random access memory (RAM), read-only memory (ROM), electronically-erasable programmable ROM (EEPROM), flash memory, or the like, or a combination thereof. Memory 64 may store program instructions that, when executed by processor 58, cause the processor to perform the functions ascribed to it herein. For example, memory 64 may store instructions for processor 58 to execute in support of control of wireless telemetry interface 68 and control of, and processing of information obtained from implantable ICP monitor 12. Memory 64 may include separate memories for storage of instructions and archived ICP information.

Telemetry interface 68 may include a wireless radio frequency (RF) receiver to permit reception of information transmitted by implanted ICP monitor 12. In some embodiments, ICP monitor 12 may be equipped for bi-directional communication, and may be responsive to commands transmitted via telemetry interface 68. In each case, telemetry interface 68 includes an antenna, in the form of shroud-like element 16, which is located proximate to a patient's head to ensure reliable telemetry.

Inductive power generator 66 applies current to shroud-like element 16 to support inductive power transfer to implanted ICP monitor 12. Although energy transfer between shroud-like element 16 and ICP monitor 12 may be relatively inefficient, external power/telemetry unit 52 preferably is coupled to a line power supply. As an example, inductive power generator 66 may drive shroud-like element 16 with a high frequency, ac signal having an amplitude sufficient for reliable telemetric energy transfer. In response to the ac signal, shroud-like element 16 transmits inductive energy to power ICP monitor 12. Telemetric energy transfer for implantable monitors is well known in the art.

Hence, external power/telemetry unit 52 enables ICP monitor 12 to be operated passively. In other words, all of the power for operation of ICP monitor 12 is provided by external power/telemetry unit 52. Yet, in accordance with the invention, shroud-like element 16 permits the power from external power/telemetry unit 52 to be coupled to ICP monitor on a continuous basis. In this manner, ICP measurements can be obtained on a substantially continuous or periodic basis, as desired.

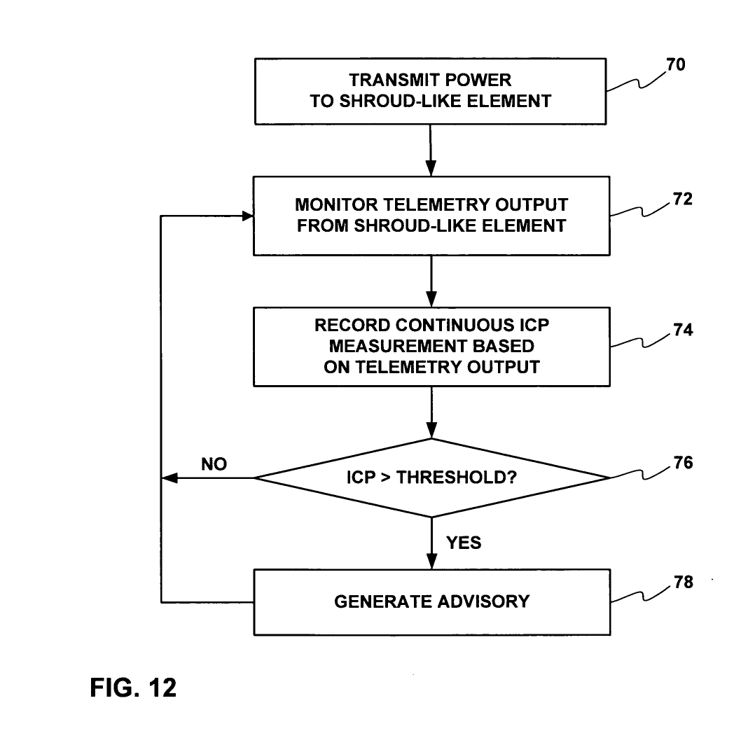

FIG. 12 is a flow diagram illustrating an ICP monitoring method in accordance with an embodiment of the invention. As shown in FIG. 12, external power/telemetry unit 52 transmits power to shroud-like element 16 to generate an electromagnetic field for transfer of energy from the shroud-like element to implantable ICP monitor 12 (70). External power/telemetry unit 52 then monitors telemetry output from shroud-like element 16 (72), which serves as a telemetry antenna for signals transmitted by ICP monitor 12. External power/telemetry unit 52 records a continuous record of ICP measurements based on the telemetry output of shroud-like element 16 (74).

In some embodiments, external power/telemetry unit 52 may invoke advisory levels to provide a care-giver with an indication when levels indicated by the measurement signals exceed a threshold level or deviate from a particular range. For example, external power/telemetry unit 52 may compare the ICP measurement to a threshold and, if the ICP measurement exceeds the threshold (76), generate an advisory (78), which may be in the form of a visual or audible alarm, alert or other conspicuous message. For example, the threshold level may be selected to alert a care-giver to the presence of ICP levels that could endanger a patient's health.

Accordingly, the ability to obtain ICP measurements on a continuous or periodic basis allows a care-giver to obtain a valuable body of information, and permits the care-giver to detect potentially dangerous ICP levels. Consequently, an ICP monitoring system 10 as described herein may contribute to improved patient care. At the same time, a shroud-like element 16 can be constructed in a manner that provides the patient with comfort and convenience.

The preceding specific embodiments are illustrative of the practice of the invention. It is to be understood, therefore, that other expedients known to those skilled in the art or disclosed herein may be employed without departing from the invention or the scope of the claims.

In the claims, means-plus-function clauses are intended to cover the structures described herein as performing the recited function and not only structural equivalents but also equivalent structures. Thus, although a nail and a screw may not be structural equivalents in that a nail employs a cylindrical surface to secure wooden parts together, whereas a screw employs a helical surface, in the environment of fastening wooden parts a nail and a screw are equivalent structures.

Many embodiments of the invention have been described. Various modifications may be made without departing from the scope of the claims. These and other embodiments are within the scope of the following claims.

1. A system for sensing intracranial pressure (ICP), the system comprising:
an implantable ICP monitor for implantation in a head of a patient;
an inductive power transmitting element sized to extend over at least a substantial portion of the head of the patient and inductively power the ICP monitor; and
an external monitor to receive a transmitted ICP signal from the ICP monitor.

2. The system of claim 1, wherein the inductive power transmitting element defines an opening to receive the head of the patient, the inductive power transmitting element including an inductive coil to inductively power the ICP monitor.

3. The system of claim 1, wherein the external monitor is coupled to receive the transmitted ICP signal from the inductive power transmitting element.

4. The system of claim 1, wherein the inductive power transmitting element includes a shroud-like element defining an opening to receive the head of the patient.

5. The system of claim 4, wherein the shroud-like element has a hemispherical shape.

6. The system of claim 1, wherein the external monitor includes a power generator to drive the inductive power transmitting element.

7. The system of claim 1, wherein the external monitor includes a display to present information based on the transmitted ICP signal.

8. The system of claim 1, wherein the inductive power transmitting element powers the ICP monitor over a substantially continuous period of time to cause the ICP monitor to transmit the ICP signal on a substantially continuous basis.

9. The system of claim 1, wherein the inductive power transmitting element is coupled to a support platform for the patient.

10. The system of claim 1, wherein the ICP monitor includes an inductive coil to receive power from the inductive power transmitting element, a pressure sensor, a telemetry interface to transmit the ICP signal, and a power generation circuit to convert the power into operating power for the pressure sensor and the telemetry interface.

11. A system for sensing intracranial pressure (ICP), the system comprising:
an inductive power transmitting element sized to extend over at least a substantial portion of a head of the patient and inductively power an ICP monitor implanted in the head of the patient;
an external monitor to receive the transmitted ICP signal from the ICP monitor.

12. The system of claim 11, wherein the inductive power transmitting element defines an opening to receive the head of the patient, the frame including an inductive coil to inductively power the ICP monitor.

13. The system of claim 11, wherein the external monitor is coupled to receive the transmitted ICP signal from the inductive power transmitting element.

14. The system of claim 11, wherein the inductive power transmitting element includes a shroud-like element defining an opening to receive the head of the patient.

15. The system of claim 11, wherein the external monitor includes a power generator to drive the inductive power transmitting element.

16. The system of claim 11, wherein the inductive power transmitting element powers the ICP monitor over a substantially continuous period of time to cause the ICP monitor to transmit the ICP signal on a substantially continuous basis.

17. A system for sensing intracranial pressure (ICP), the system comprising:
means, extending over a substantial portion of the head of a patient, for inductively powering an ICP monitor implanted in the head of the patient; and
means for receiving the transmitted ICP signal from the ICP monitor via the means for inductively powering the ICP monitor.

18. The system of claim 17, wherein the means for inductively powering the ICP monitor defines an opening to receive the head of the patient, the frame including an inductive coil to inductively power the ICP monitor.

19. The system of claim 17, further comprising means for powering the inductive power transmitting element.

20. The system of claim 17, wherein the means for inductively powering the ICP monitor powers the ICP monitor over a substantially continuous period of time to cause the ICP monitor to transmit the ICP signal on a substantially continuous basis.

21. The system of claim 17, wherein the means for receiving the transmitted ICP signal includes the means for inductively powering the ICP monitor as an antenna and a means for monitoring an output of the means for inductively powering the ICP monitor.

22. A method for sensing intracranial pressure (ICP), the method comprising:
powering an inductive power transmitting element sized to extend over at least a substantial portion of the head of the patient to inductively power an ICP monitor implanted in the head of the patient; and
receiving an ICP signal transmitted by the ICP monitor via an output of the inductive power transmitting element.

23. The method of claim 22, wherein the inductive power transmitting element defines an opening to receive the head of the patient, the frame including an inductive coil to inductively power the ICP monitor.

24. The method of claim 22, wherein the inductive power transmitting element includes a shroud-like element defining an opening to receive the head of the patient.

25. The method of claim 24, wherein the shroud-like element has a hemispherical shape.

26. The method of claim 24, further comprising powering the inductive power transmitting element to power the ICP monitor over a substantially continuous period of time to cause the ICP monitor to transmit the ICP signal on a substantially continuous basis.

27. The method of claim 22, wherein the inductive power transmitting element is coupled to a support platform for the patient.
